(19) United States
(12) Patent Application Publication
(10) Pub. No.: US 2015/0119459 A1
(43) Pub. Date: Apr. 30, 2015

(54) DERMAL-APPROPRIATE COMPOSITIONS AND METHODS OF USE

(71) Applicant: NBIP, LLC, Plano, TX (US)

(72) Inventors: Burt R. Sookram, Palm Harbor, FL (US); John W. Veenstra, Frisco, TX (US)

(21) Appl. No.: 14/396,543

(22) PCT Filed: Mar. 12, 2013
(86) PCT No.: PCT/US13/30588

§ 371 Date: Oct. 23, 2014

Related U.S. Application Data
(60) Provisional application No. 61/636,948, filed on Apr. 23, 2012.

(51) Int. Cl. A61K 8/67; A61Q 19/00

(52) U.S. Cl. CPC A61K 8/676; A61Q 19/00; A61K 2800/83
USPC 514/474

(57) ABSTRACT

This invention relates to mild or weak acids and salt mixtures, such as Ascorbic acid, that when mechanically enhanced become dermal-appropriate, thereby allowing high levels of these compositions to be used in health-care, medical, pharmaceutical, nutraceutical and cosmoceutical products.

### CROSS-REFERENCES TO RELATED APPLICATIONS

This Application claims the benefit under 35 U.S.C. §119(e) of U.S. Provisional Patent Application Ser. No. 61/636,948 filed Apr. 23, 2012, which is incorporated herein by reference in its entirety as if fully set forth herein.

### FIELD OF THE INVENTION

This invention relates to mild or weak-acid/salt compositions, such as Ascorbic Acid, that when mechanically enhanced become dermal-friendly thereby allowing high levels of these mixtures to be used in healthcare, medical, pharmaceutical, nutraceutical and cosmeceutical products.

### BACKGROUND OF THE INVENTION

Ascorbic acid (C₆H₈O₆) is a naturally occurring organic compound with antioxidant properties. It is usually a white solid which is easily soluble in water producing a mildly acidic solution. It behaves as a carboxylic acid with the electrons in the double bonded hydroxyl group lone pair and the carbonyl double bond forming a conjugated system. The hydroxyl group in ascorbic acid is much more acidic than typical hydroxyl groups.

A mild or weak acid is an acid that dissociates incompletely. It does not release all of its hydrogens in a solution, donating only a partial amount of its protons to the solution. These acids have higher pKa than strong acids, which release all of their hydrogen atoms when dissolved in water. Weak acids and bases are only partially ionized in their solutions. A weak acid is incapable of getting completely ionized in a water solution and releases a lesser amount of hydrogen ions, compared to strong acids. Due to the peculiar nature of chemical bonds in weak acids, protons or Hydrogen ions are not released easily in an aqueous solution.

The acid dissociation constant Kₐ is generally used in the context of acid-base reactions. The numerical value of Kₐ is equal to the concentration of the products divided by the concentration of the reactants, where the reactant is the acid (HA) and the products are the conjugate base and H. These mild or weak acids are characterized by a Kₐ ranging from 10⁻³ to 10⁻¹⁰. The acid dissociation constant of any weak acid can be calculated from the concentration of hydrogen ions in its aqueous solution.

In order to lose a proton, it is necessary that the pH of the system rise above the pKa of the protonated acid. The decreased concentration of H³⁰ in that primary solution shifts the equilibrium towards the conjugate form which is the deprotonated form of the acid. In lower pH solutions, which are mixtures that are more acidic, there is a high enough H⁺ concentration in the solution to cause the acid to remain in its protonated form.

As with strong acids such as sulfuric acid (H₂SO₄) that are very corrosive, some mild acids in stronger concentrations can also be corrosive making them both problematic and potentially dangerous. Further, although the outermost layer of the epidermis (skin) includes a layer of dead cells that protect the living cells beneath, if the mild acid is sufficiently concentrated, it can destroy that layer of dead skin cells, exposing the more vulnerable dermal cells beneath. This property renders both strong and mild acids in concentrated amounts generally unsuitable for use in applications where it will come into contact with skin when used in nutraceutical, cosmoceutical and other healthcare applications.

The non-dermal nature of an acid can be controlled by diluting it in sufficient amounts of water. However, the volume of the diluted acid needed to provide sufficient H₃O⁺ or OH⁻ makes the end-product ineffective. Alternatively, the acid may be combined with an appropriate salt. For example, if water, ascorbic acid, and ammonium ascorbate are combined in solution, the intermolecular interactions between the H₃O⁺, NH₄⁺, and C₆H₇O₆⁻ are sufficient to keep the C₆H₇O₆⁻ from irritating or destroying skin. However, these same intermolecular interactions leave the solution insufficiently reactive to affect the cell membranes.

What is needed, therefore, is a composition that is reactive like an acid, yet can be safely stored and used in medical, pharmaceutical, nutraceutical, cosmoceutical and other healthcare applications without causing any skin irritation or damage, and can be safely stored without a corrosive effect.

### SUMMARY OF THE INVENTION

An embodiment of the invention is directed toward using a pulsed direct current to energize a solution of a concentrated weak acid, a salt, and water, such that the resulting composition does not have the expected corrosive or caustic properties and does not have the expected skin-damaging properties, yet is sufficiently reactive to affect hydrogen bonds. A further embodiment of the invention is directed at the resulting composition.

### BRIEF DESCRIPTION OF THE DRAWINGS

FIG. 1 is a flowchart of a mechanical enhancement process using a mild acid such as ascorbic acid in accordance with an embodiment of the invention;

FIG. 2 is a flowchart of a mechanical enhancement process using a mild acid such as ascorbic acid in accordance with an embodiment of the invention;

FIG. 3 is a block diagram of equipment used in performing the mechanically enhanced process; and

FIG. 4 is a block diagram of equipment used in performing the mechanically enhanced process.

### DETAILED DESCRIPTION OF EXEMPLARY EMBODIMENTS

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a carrier” includes mixtures of two or more such carriers, and the like.

Ranges may be expressed herein as from “about” one particular value, and/or to “about” another particular value. When such a range is expressed, another aspect includes from the one particular value and/or to the other particular value. Similarly, when values are expressed as approximations by use of the antecedent “about,” it will be understood that the particular value forms another aspect. It will be further understood that the endpoints of each of the ranges are significant both in relation to the other endpoint and independently of the other endpoint.

“Admixture” or “blend” as generally used herein means a physical combination of two or more different components

“Hydrogen Bonds” as used herein means the electrostatic dipole moments interaction which are the strong directional forces that hold both the inorganic and organic molecules and proteins together to form a chain.

“Dermal Environment” as used herein refers to the multiple layers of skin tissue associated with either humans or animals.

“Dermal Friendly” as used herein refers to a composition that has a neutral or beneficial effect on skin tissue when applied to the outer layer of skin tissue, with no or minimal negative effects.

“Intermolecular Attractions” as used herein refers to the attractions between one molecule and a neighboring molecule.

“Optional” or “optionally” means that the subsequently described event or circumstance can or cannot occur, and that the description includes instances where the event or circumstance occurs and instances where it does not.

“pH” as used herein is a number that is measured in a 1% solution of a weak acid or a weak acid/salt mixture, with the remainder of the solution being water.

“1% solution” is used herein is defined as 1 part of the weak acid or a weak acid/salt mixture and 99 parts of water.

“Pulse or pulsing” as used herein refers to as a single application of a direct current to a solution. Multiple pulsing or pulses make up a pulsing event.

“Pulsing Event” as used herein refers to a series of pulses followed by a resting period. There can be multiple pulsing events in a single iteration of the inventive method.

“Weak Acids” is used herein to refer to any acid that are mildly corrosive and normally do not affect skin, and are referred to as organic acids or natural acids and have a pH ranging from 3.5 to 6.9 at 100% concentration.

“Weak Acid/Salt” as used herein refers to any salt that will effectively combine with the chosen weak acid.

By “sufficient amount” and “sufficient time” means, an amount and time needed to achieve the desired result or results.

A “weight percent” of a component, unless specifically stated to the contrary, is based on the total weight of the formulation or composition in which the component is included.

Weak Acid Embodiments

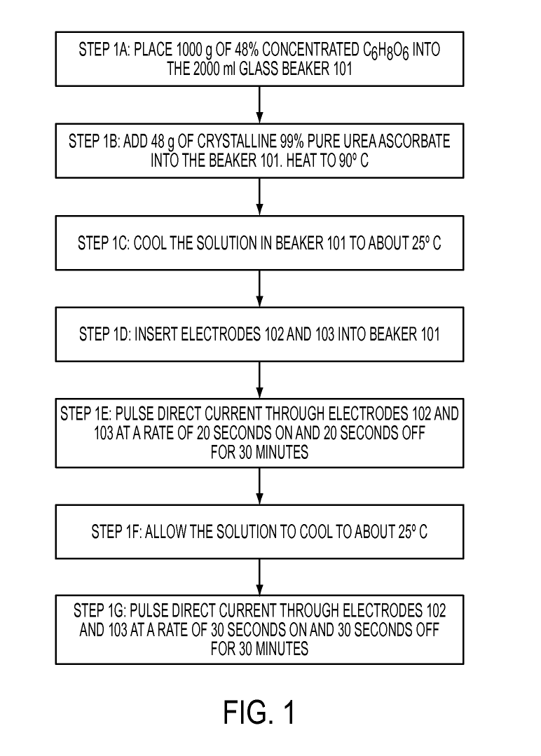

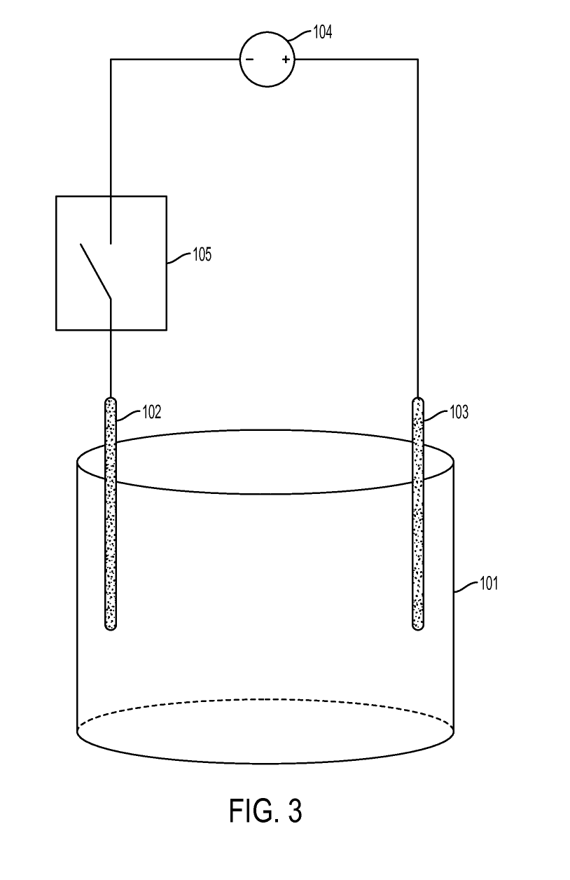

FIG. 1 shows a flowchart of a preferred embodiment of the inventive process using a weak acid, namely ascorbic acid. In step 1A, about 1000 grams of a 45% ascorbic acid/water mixture is placed into a 2000 ml glass beaker 101. In step 1B, about 48 grams of crystalline 99% pure urea ascorbate is added to beaker 101. After the addition of the ascorbate, the mixture is heated to about 90° C. for 15 minutes to allow the mixture to completely dissolved. The mixture was stirred regularly. In step 1C, once all of the ascorbate salts are dissolved, the solution is allowed to cool to between 23-25° C. At this point, the solution contains a mix of hydronium and ammonium cations, and hydroxide and ascorbate anions. The measured conductivity was less than 150 mV, the measured proton count was about 1.0×10²⁴, and the pH was about 2.9 to 3.2. Where numeric values or ranges of values of conductivity, proton count, or pH of the solution are disclosed, the conductivity measurements are made on the pure solution, proton count measurements are made on a sample of the pure solution, and pH measurements are made on a 100% concentration of the solution. Based on observations, it is believed that at this stage of the process, the attractions between the oppositely charged ions in the solution make it more dermal-friendly than untreated ascorbic acid. However, the solution lacks the qualities that would make it sufficiently reactive to disrupt hydrogen bonds. In step 1D, two electrodes 102 and 103 are placed into the beaker 101 at opposite sides of the beaker, away from the walls of the beaker, and partially submerged in the solution. The electrodes 102 and 103 are connected to a direct current power source 104 with an inline switch 105. Switch 105 could be a manual switch, a strobe light controller, laboratory voltage pulser, or comparable circuit to provide the direct current pulses. FIG. 3 shows a block diagram of the equipment used in an embodiment of the inventive process. In step 1E, a 3 amp direct current at 10 volts is pulsed through the solution between the electrodes for about 30 minutes, where the pulsing period is about 20 seconds on and 20 seconds off. After allowing the solution to cool in Step 1F, the measured conductivity was about 500 mV, the measured proton count was about 9×10²⁴, and the pH was about 3-3.3.

In Step 1G, after the first period of pulsing the current through the solution, and after the solution had cooled to between 23° C. and 25° C., a second round of pulsing is performed, comparable to the first and lasting a length of about 30 minutes, where the pulsing period was about 20 seconds on and 20 seconds off. After this second round of pulsing, the measured conductivity was about 500 mV, the measured proton count was about 9×10²⁴, and the pH was about to 3 to 3.3. Over time (several months) the conductivity did not measurably decrease (data not shown), suggesting that the second round of pulsing not only increased the reactivity but added stability to the composition. While not being bound to specific theories, based on empirical observations, it is believed that the controlled application of direct current increases the lengths of the bonds in the polar molecules, leading to higher reactivity. Further, because the current is pulsed, it does not interfere with the intermolecular bonds between the oppositely-charged ions and in fact strengthens those bonds, thus retaining and enhancing the composition's dermal-appropriate qualities. Further, because of the stability of the hydrogen bonds, when the composition is stored under non-adverse conditions (for example, away from extreme heat, light, pressure, or electromagnetic radiation), it retains its reactive and dermal-friendly qualities indefinitely. Further, consistent with observations, it is seen that when steady (non-pulsed) or alternating current is used, or higher-power current, or when the temperature is not controlled during the pulsing process, the composition did not have these enhanced reactive and dermal-appropriate qualities. This does not, however, preclude the use of other energy sources, such as sound, electricity, light, or mechanical sources, provided the application of energy does not break down the intermolecular bonding. Thus, an embodiment of the invention addresses the need for a stable composition that is reactive, like a weak acid, yet does not corrode metal or irritate skin.

In other embodiments, the concentration of the acid may be varied without affecting the general process or the characteristics of the resulting composition. However, use of too diluted a concentration may lower the ranges of conductivity and proton count in the final composition and therefore limit its usefulness. The efficacy of a given concentration of acid can be determined from routine experimentation based on the embodiments disclosed herein.

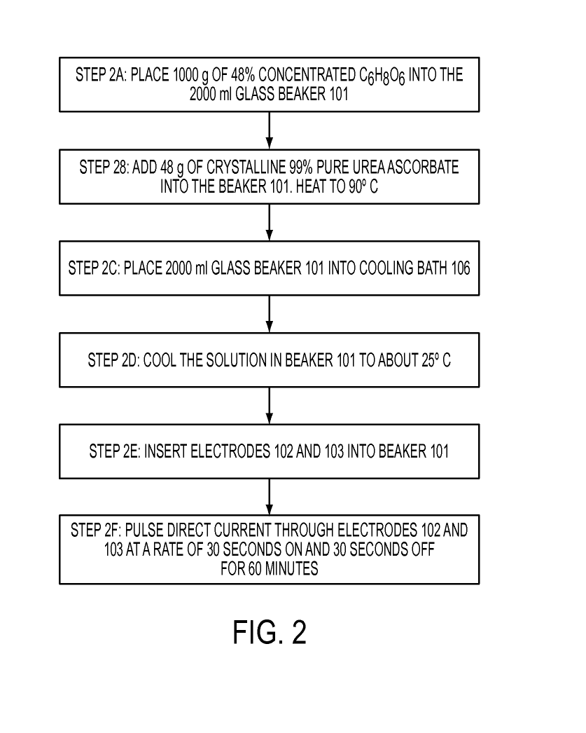

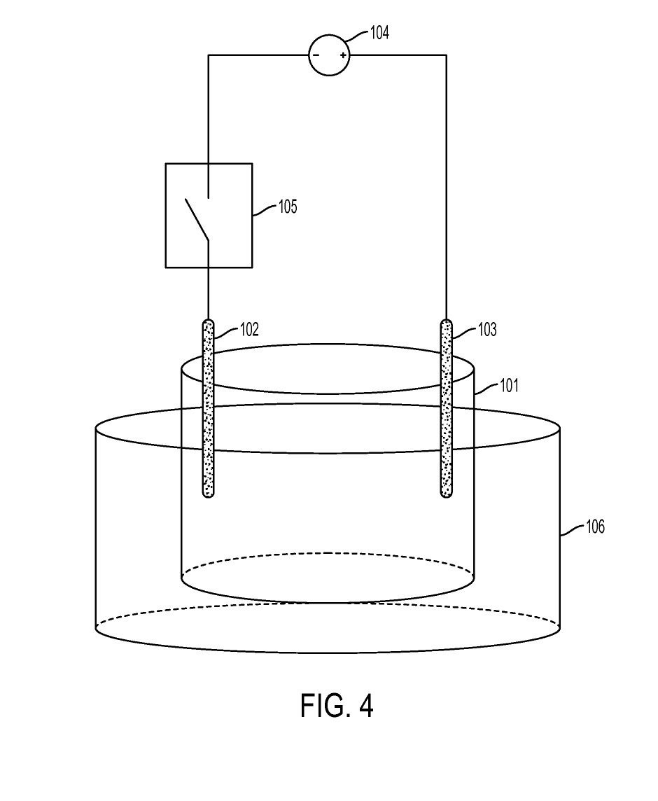

In the embodiment described above, pulsing of the solution occurs in two steps. This is to help control the temperature of the solution, as it has been found that excessive heat appeared to break down intermolecular bonds instead of simply energizing them, leading to a solution that did not have the desired properties. In other embodiments, the pulsing can occur in a single step, provided that the temperature of the solution is kept under about 25° C., using cooling techniques that are known in the art, for example, partially submersing the mixing vessel in a cooling bath, as shown in the block diagram of FIG. 4. The process described in the flowchart of FIG. 2 differs from the process of FIG. 1 in that after the C₆H₈O₆ and C₆H₇NH₃O₆ are mixed together, the beaker 101 is placed into a cooling bath 106, which maintains a relative constant temperature allowing charging, and the pulsing process to be performed in a single 60-minute step.

In other embodiments, the voltage, amperage, period, and duration of the pulsing current could be varied without adversely affecting the desired properties. Such variations could be necessitated, for example, by the size of the electrodes, the size of the beaker, and the volume of the weak acid/salt solution. In practice, we found that we could obtain the desired properties of the modified weak acid/salt solution with voltages ranging from 4 to 16 volts, currents ranging from 1 to 20 amps; pulse periods ranging from 5 to 60 seconds on and 5 to 60 seconds off, and pulsing current duration ranging from 20 to 70 minutes. In determining these ranges, we applied the pulsing current at 1 atmosphere. Varying the pressure could broaden or narrow these ranges without affecting the end results, and new effective ranges for different pressure constraints could be determined through routine experimentation.

Tables 1 and 2 below show the results of experiments performed in accordance with the inventive embodiments of the claimed invention.

A 45% solution of ascorbic acid in water having a starting pH of 3.18 was pulsed at 4 amps, 12 watts for 60 minutes in a continuous pulsing process. Table 1 below shows the results of this experiment.

TABLE 1

| Proton | | | | | |
|---|---|---|---|---|---|
| Count | | | | | |
| Before | After | | 1 hour | Final | pH after |
| Charge | charge | Initial mV | mV | Temp | charging |
| 1.70 × 10²⁴ | 9.2 × 10²⁴ | 175 | 510 | 89 C. | 3.20 |
| 1.78 × 10²⁴ | 9.6 × 10²⁴ | 170 | 505 | 91 C. | 3.18 |
| 1.85 × 10²⁴ | 1.05 × 10²⁵ | 172 | 510 | 65 C. | 3.20 |
| 1.82 × 10²⁴ | 1.01 × 10²⁵ | 180 | 500 | 65 C. | 3.24 |

In an another experiment, a 45% solution of ascorbic acid in water having a starting pH of 3.20 was pulsed in a 2 step charging process involving a first pulsing step for 30 minutes, followed by a cooling period, and a second pulsing step for 30 minutes. Table 2 below shows the results of this experiment.

TABLE 2

| Proton Count | | | 30 | 1 | | pH |
|---|---|---|---|---|---|---|
| Before | After | Initial | min | hour | Final | after |
| Charge | charge | mV | mV | mV | Temp | charging |
| 1.69 × 10²⁴ | 9.8 × 10²⁴ | 155 | 370 | 505 | 85/92 C. | 3.20 |
| 1.88 × 10²⁴ | 8.8 × 10²⁴ | 160 | 390 | 525 | 83/91 C. | 3.20 |
| 1.75 × 10²⁴ | 0.92 × 10²⁵ | 168 | 370 | 510 | 65 C. | 3.24 |
| 1.91 × 10²⁴ | 0.85 × 10²⁵ | 168 | 380 | 510 | 65 C. | 3.24 |

In certain embodiments, the ammonium ascorbate salt can be replaced with other ascorbate salts such as, for example, sodium ascorbate, potassium ascorbate, calcium ascorbate, magnesium ascorbate, aluminum ascorbate, urea ascorbate, zinc ascorbate, nickel ascorbate, lead ascorbate, copper ascorbate, ferrous ascorbate, ferric ascorbate, gold ascorbate, or comparable ascorbate salts (or combinations of ascorbate salts). The choice of one particular salt over another does not affect the general process or characteristics of the resulting composition. However, the choice of a particular salt and its purity may change the proportions of the various components used in the process, it may change the measured ranges of conductivity and proton count of the composition, and selection of a particular salt may result in the composition having useful or detrimental characteristics beyond those described here. The optimal quantities of components and length/magnitude of current pulsing for any given substitute salt can be determined from routine experimentation based on the embodiments disclosed in this patent.

In other embodiments, the ascorbic acid can be replaced with another weak acid. By way of example, the following weak acids could be used phosphoric acid (H₃PO₄), citric acid (H₃C₆H_{S}O₇), nitrous acid (HNO₂), hydrofluoric acid (HF), formic acid (HCOOH), benzoic acid(C₆H₅COOH), sorbic acid (C₆H₈O₂), acetic acid CH₃COOH), carbonic acid (H₂CO₃), boric acid (H₃BO₃), tartaric acid (C₄H₆O₆), salicylic acid (C₇H₆O₃), hypochlorous acid (HClO), hydrocyanic acid (HCN) or any acid with a pH of between 3.5 to 6.9. The choice of one particular acid over another does not affect the general process or characteristics of the resulting composition; however, the choice of a particular weak acid and its purity may change the proportions of the various components used in the process, it may change the measured ranges of conductivity and proton count of the composition, and selection of a particular weak acid may result in the composition having useful or detrimental characteristics beyond those described here. The optimal quantities of components and length/magnitude of current pulsing for any given substitute weak acid can be determined from routine experimentation based on the embodiments disclosed herein.

In selecting substitute weak acid and/or salt components, the following guidelines have been found to be true. First, we found that ammonium salts were preferable over non-ammonium salts. While not binding ourselves to specific theories, we believe that because of its size and polarity, the NH₄, tends to form relatively stable intermolecular bonds with negatively-charged anions (for example, C₆H₈O₆₋), even after the direct current pulsing step(s). Thus the composition remains non-corrosive and dermal-friendly after charging, but the increased polarity makes the composition sufficiently reactive to disrupt other hydrogen bonds, such as those found in cell membranes. This preference for an ammonium salt notwithstanding, non-ammonium salts which dissociate into cations that behave similarly to NH₄₊ may prove suitable, especially in applications where a non-ammonium salt brings additional benefits.

In certain embodiments, selecting a salt with the same or similar anion to the weak acid for example, C₆H₇O₆₋ is preferable to those with dissimilar anions. With a more homogenous solution, it is believed there will be fewer undesirable side reactions. However, selecting a weak acid and salt with dissimilar anions may nonetheless prove suitable, especially in applications where the dissimilar anion of the salt brings additional benefits.

Thus, using these guidelines, by way of example and not limitation, the following weak acids could be used: : phosphoric acid (H₃PO₄), citric acid (H₃C₆H_{S}O₇), nitrous acid (HNO₂), hydrofluoric acid (HF), formic acid (HCOOH), benzoic acid(C6H5COOH), sorbic acid (C₆H₈O₂), acetic acid CH₃COOH), carbonic acid (H₂CO₃), boric acid(H₃BO₃), tartaric acid (C₄H₆O₆), salicylic acid (C₇H₆O₃), hypochlorous acid (HC1O), hydrocyanic acid (HCN) and any organic acid.

In certain embodiments, the use of the modified weak acid/salt composition causes the cell membranes to be more susceptible to the interruption of the hydrogen bonds while at the same time being dermal appropriate and suitable for use in situations where it is in contact with the skin.

The optimal quantities of components, length, sequence and magnitude of mechanical enhancement for any given substitute mild acid/salt can be determined from routine experimentation based on the embodiments disclosed in this patent. While specific embodiments have been illustrated and described, numerous modifications are possible without significantly departing from the spirit of the invention and the scope of protection is only limited by the scope of the accompanying claims.

What is claimed is:

1. A method for producing a modified weak acid/salt solution, comprising:
subjecting a starting solution of a weak acid and a salt to at least one pulsing event, wherein said pulsing event comprises at least one pulse of direct current; and
modifying the starting solution to produce a modified solution having a higher conductivity and higher proton count than the starting solution.

2. The method of claim 1, wherein the pulse ranges from 1 to 20 amps at 4 to 16 volts and lasts between 5 to 60 seconds.

3. The method of claim 1, wherein the pulsing event comprises passing at least one additional pulse of direct current through the starting solution.

4. The method of claim 3, wherein the time interval between pulses in a pulsing event ranges from 5 to 60 seconds.

5. The method of claim 1 further comprising subjecting the solution to an additional pulsing event.

6. The method of claim 5, wherein the solution is subjected to pulsing for a length of time ranging from 20 to 70 minutes.

7. The method of claim 1, wherein the number of pulsing events ranges from 1 to 5.

8. The method of claim 1 further comprising cooling the starting solution to between 23° C. and 25° C. after each pulsing event.

9. The method of claim 1, wherein the modified solution has a conductivity of between 500 and 550 mV, a proton count of between 1×10²⁴ and 1.5×10²⁵ and a pH of between 3 and 4.

10. The method of claim 1, wherein the weak acid is ascorbic acid that is about 70% concentrated, the salt is ammonium ascorbate and is about 99% pure, and the weak acid and salt are combined at about a 6 to 1 ratio by weight.

11. The method of claim 1, wherein the alternate s alt is selected from the group consisting of sodium ascorbate, potassium ascorbate, calcium ascorbate, magnesium ascorbate, aluminum ascorbate, zinc ascorbate, urea a s c orb ate, nickel ascorbate, lead ascorbate, copper ascorbate, ferrous ascorbate, ferric ascorbate and combinations thereof.

12. The method of claim 1, wherein weak acids are selected from the group consisting of formic acid, acetic acid, trichloroacetic acid, hydrofluoric acid, hydrocyanic acid, nitrous acid, acetylsalicylic acid, benzoic acid, phenol, and any acid having a pH of between 3.5 and 6.9.
